(19) United States
(12) Patent Application Publication
(10) Pub. No.: US 2026/0083455 A1
(43) Pub. Date: Mar. 26, 2026

(54) IMPLANT FOR SINUS CAVITY RECONSTRUCTION

(71) Applicant: SinUse Corp., West Hollywood, CA (US)

(72) Inventors: Gabriel Zada, Los Angeles, CA (US); Seyed Mohammadreza Ghodsi, West Hollywood, CA (US)

(21) Appl. No.: 19/112,178

(22) PCT Filed: Sep. 14, 2023
(86) PCT No.: PCT/US2023/074244

§ 371 Date: Mar. 14, 2025

Related U.S. Application Data
(60) Provisional application No. 63/407,102, filed on Sep. 15, 2022.

(51) Int. Cl. A61B 17/12; A61B 17/00

(52) U.S. Cl. CPC A61B 17/12104; A61B 17/12181; A61B 2017/00862; A61B 2017/00955; A61B 17/12031

(57) ABSTRACT

An implant for a sinus cavity that can have a main body portion and a second body portion coupled with the main body portion. In some embodiments, the second body portion can extend at an angle away from the main body portion. The implant can be sized and configured for implantation in the frontal sinus. A size and a shape of the implant can be similar to a size and a shape of the frontal sinus of a patient that the implant will be implanted into.

WO

### INCORPORATION BY REFERENCE TO ANY PRIORITY APPLICATIONS

This application claims priority to U.S. Provisional Ser. No. 63/407,102, filed Sep. 15, 2022, which is incorporated herein by reference as if fully set forth herein. Any and all applications for which a foreign or domestic priority claim is identified in the Application Data Sheet as filed with the present application are hereby incorporated by reference under 37 CFR 1.57.

### BACKGROUND OF THE DISCLOSURE

### Field of the Disclosure

Some embodiments of the present disclosure relate to a novel implant for frontal sinus cavity reconstruction and obliteration. For example and without limitation, some embodiments of the sinus implant disclosed herein can be a 3D printed sinus implant that can be inserted or otherwise implanted into the frontal sinus cavity after frontal craniotomy operations in order to facilitate frontal sinus obliteration and avoid post-surgery complications.

### Description of the Related Art

The frontal sinus typically consists of two air-filled volumes situated behind the right and left part of the forehead and above the eyes and nasal cavity. The left and right frontal sinus are typically separated by the frontal septum in the sagittal plane. In adults, both frontal sinuses are roughly pyramid shaped. In the frontal sinus floor, there is a conically tapering constriction, the frontal sinus infundibulum. It leads to the smallest constriction of the passage to the frontal sinus, the frontal ostium. It is the opening of the frontal sinus to the natural frontal sinus outflow tract (frontal recess). The frontal sinus outflow tract leads downward around the agger nasi cell into the nasal cavity. From here, there exists a path which leads past the middle turbinate to the nostril and the outside air. In normal mucociliary flow, the secretions are transported laterally over the superior mucosa, and move back over the inferior mucosa before finally draining into the frontal recess.

There is no established method for frontal sinus repair following trauma, infection, or iatrogenic exposure during surgery for a variety of indications. Current strategics for frontal sinus reconstruction during a craniotomy setting involve multilayer tissue closure including allograft, autograft such as muscle and/or fat, and a pericranial flap as well as a variety of foreign potential packing materials. In many cases such as trauma or tumor cases, additional tissue is unavailable and harvesting of autologous reconstructive material such as fat or fascia can be detrimental to the patient with additional pain or risk for infection.

Existing strategies for frontal sinus repair, reconstruction or obliteration have a known failure rate that may result in patient morbidity or mortality, cerebrospinal fluid leakage, infection and meningitis, pneumocephalus, neurological injury and death, and no standardized method or devices are currently available specifically designed for frontal sinus reconstruction.

### SUMMARY OF SOME EXEMPLIFYING EMBODIMENTS

The embodiments disclosed herein each have several aspects, no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for obliterating sinus cavities.

The following disclosure describes non-limiting examples of some embodiments. Other embodiments of the disclosed systems and methods may or may not include the features described herein. Moreover, disclosed advantages and benefits may apply only to certain embodiments of the sinus implant disclosed herein and should not be used to limit the disclosure.

Disclosed herein are embodiments of a device for treating a sinus cavity or condition. Also disclosed herein are embodiments of an implant for a sinus cavity that can include, in some embodiments, a main body portion, a second body portion coupled with the main body portion, wherein the implant is sized and configured for implantation in the frontal sinus, and wherein a size and a shape of the implant is similar to a size and a shape of the frontal sinus of a patient or patients that the implant will be implanted into.

Any embodiments of the devices, systems, and methods disclosed herein can include, in additional embodiments, one or more of the following features, components, and/or details, in any combination with any of the other features, components, and/or details of any other embodiments of the devices, systems, and methods disclosed herein: wherein in some embodiments of the implant the second body portion can extend at an angle away from the main body portion; wherein in some embodiments of the implant the second body portion can have a tapering cross-sectional shape along a length of the second body portion such that a cross-sectional size of the second body portion can be smaller at a distal end portion of the second body portion than at a proximal portion of the second body portion; wherein in some embodiments of the implant the proximal portion of the second body portion can be coupled with the main body portion; wherein the second body portion can extend away from the main body portion at an approximately 70 degree angle relative to an approximate centerline of the main body portion; wherein in some embodiments of the implant the second body portion can extend away from the main body portion at an angle that is from 60 degrees to 80 degrees relative to an approximate centerline of the main body portion; wherein in some embodiments of the implant the second body portion can extend away from the main body portion at an angle that is from approximately 60 degrees to approximately 80 degrees relative to an approximate centerline of the main body portion; wherein some embodiments of the structure can be printed in a thermoplastic polymer material; wherein the thermoplastic polymer material can include one or more of polyethylene, high density polyethylene (HDPE), polymethyl methacrylate (PMMA), or polylactide (PLA), or be a thermoset crosslinked polymer that can include one or more of polyethylene glycol (PEG), polydimethylsiloxane (PDMS), or polydioxanone (PDS); wherein some embodiments of the implant can be made from a single monolithic piece of material; wherein some embodiments of the implant can be made from an elastic material; wherein some embodiments of the implant can be sized and shaped to fill or obliterate the gap and dead space or potential space that makes up the frontal sinus between the forehead, frontal bone, sinus septum, and frontal infundibulum; wherein the shape and size of some embodiments of the implant can be based on extensive analysis of the morphology of the frontal sinus in normal individual and patients using a collection of head scans of adult males and females; wherein a width of some embodiments of the implant from an anterior to a posterior surface of the implant can be from 15 mm or approximately 15 mm to 20 mm or approximately 20 mm, or any values between 15 mm and 20 mm; wherein an anterior-posterior dimension of some embodiments of the implant can be from 15 mm or approximately 15 mm to 20 mm or approximately 20 mm, or any values between 15 mm and 20 mm; wherein a width from an anterior to a posterior surface of some embodiments of the implant can be 15 mm or approximately 15 mm; wherein a width from an anterior to a posterior surface of some embodiments of the implant can be 18 mm or approximately 18 mm; wherein a width from an anterior to a posterior surface of some embodiments of the implant can be 20 mm or approximately 20 mm; wherein a length of some embodiments of the implant can be from 40 mm or approximately 40 mm to 70 mm or approximately 70 mm, or any values between 40 mm and 70 mm; wherein a length of some embodiments of the implant can be 40 mm or approximately 40 mm; wherein a length of some embodiments of the implant can be 55 mm or approximately 55 mm; wherein a length of some embodiments of the implant can be 70 mm or approximately 70 mm; wherein a height of some embodiments of the implant can be from 15 mm or approximately 15 mm to 30 mm or approximately 30 mm, or any values between 15 mm and 30 mm; wherein a height of some embodiments of the implant can be 15 mm or approximately 15 mm; wherein a height of some embodiments of the implant can be 25 mm or approximately 25 mm; wherein a height of some embodiments of the implant can be 30 mm or approximately 30 mm; wherein some embodiments of the implant can be prism-shaped; wherein some embodiments of the implant can have one or more protrusions on each of an anterior surface and a posterior surface of the implant; wherein some embodiments of the implant can be prism-shaped and have one or more protrusions on each of an anterior surface and a posterior surface, wherein, in some embodiments of the implant, the protrusions can be trimmable or trimmed by hand to better confirm the implant to a patient's sinus cavity; wherein some embodiments of the implant can have a tapered adjunction to completely fill the frontal infundibulum and/or the frontal recess in a water-tight fashion, or to substantially completely fill (e.g., fill 90% or more of the space) the frontal infundibulum and/or the frontal recess; wherein some embodiments of the implant may be made from a putty like material or a material that is shapable and may resemble a putty or moldable material; wherein the implant can be printed in different layers to have a changing gradient of hardness from a core portion of the implant to an outer surface of the implant that decreases in hardness; wherein a core portion of some embodiments of the implant can be made from a first material and an outer portion of some embodiments of the implant can be made from a second materials this can be different than the first material; wherein a core portion of some embodiments of the implant can be made from one or more of polyethylene, high density polyethylene (HDPE), polymethyl methacrylate (PMMA), polylactide (PLA), polyethylene glycol (PEG), polydimethylsiloxane (PDMS), polydioxanone (PDS); wherein an outer portion of the implant can be made from one or more of polyethylene, high density polyethylene (HDPE), polymethyl methacrylate (PMMA), polylactide (PLA), polyethylene glycol (PEG), polydimethylsiloxane (PDMS), polydioxanone (PDS); wherein a core portion of some embodiments of the implant can be harder than an outer portion of the implant; wherein some embodiments of the implant can be PEGylated to improve a compliance of the implant for improved fit, hydrophobicity, and drug delivery applications; wherein some embodiments of the implant can be configured to expand in situ by 5% or approximately 5% to 20% or approximately 20% by volume, or any values between 5% and 20%, after implantation in a patient's sinus cavity; wherein the implant is configured to expand in situ by 5% or approximately 5% to 10% or approximately 10% by volume, or any values between 5% and 10% after implantation in a patient's sinus cavity; wherein some embodiments of the implant can be configured to expand in situ by at least 10% by volume after implantation in a patient's sinus cavity; wherein a core portion of the implant is 3D printed from a PE, HDPE, PMMA, or PLA with elastic modulus above 500 MPa; wherein in some embodiments of the implant an outer portion of the implant can be 3D printed from PDMS, PEG, or PDS; wherein an outer portion of some embodiments of the implant has an elastic modulus from 2 MPa to 5 MPa, or from approximately 2 MPa to 5 MPa; wherein an outer portion of some embodiments of the implant can have an elastic modulus of above 5 MPa; wherein an outer portion of some embodiments of the implant can have an elastic modulus 1 MPa or lower; wherein some embodiments of the implant can be printed from a biocompatible thermoplastic polymer; wherein some embodiments of the implant can be printed in a thermoset crosslinked polymer; wherein some embodiments of the implant can be printed in PEG or PDMS; wherein some embodiments of the implant can be printed in different layers to have a gradient of hardness that decreases from a middle portion of the implant to a surface portion of the implant; wherein some embodiments of the implant can be configured to elute antibiotics and/or steroids or other compounds to suppress inflammation or infection; wherein some embodiments of the implant can be prefabricated to a plurality of shapes, sizes and volumes to accommodate common morphologies of the frontal sinus in human patients; wherein some embodiments of the implant can be expandable, hydrophobic, and drug cluting; wherein some embodiments of the implant can be biodegradable or bioabsorbable; and/or wherein the implant can be a permanent implant. In any of the embodiments of the implant, method, or kit, set, or tray disclosed herein, the implant can be tear shaped or approximately tear shaped, or pear shaped or approximately pear shaped.

Also disclosed herein are embodiments of a method of making a sinus implant for a frontal sinus that can, in some embodiments, include reviewing imaging data (e.g., CT scan or MRI) of a frontal sinus region from a plurality of patients, forming a first frontal sinus implant for a first range of patients, the first frontal sinus implant having a first size and that can include a main body portion and a second body portion extending at an angle away from the main body portion. In some embodiments, the first frontal sinus implant can be shaped to approximately match a shape of the frontal sinus.

Any embodiments of the devices, systems, and methods disclosed herein can include, in additional embodiments, one or more of the following features, components, and/or details, in any combination with any of the other features, components, and/or details of any other embodiments of the devices, systems, and methods disclosed herein: wherein the method can include forming a second frontal sinus implant for a second range of patients, wherein the second frontal sinus implant can be larger than the first frontal sinus implant and can be sized and configured to approximately match at least a frontal sinus of the second range of patients; wherein the method can include forming a third frontal sinus implant for a second range of patients; wherein the second frontal sinus implant can be larger than the second frontal sinus implant and can be sized and configured to approximately match at least a portion of the frontal sinus of the third range of patients; and/or wherein the method can include sterilizing the sinus implant with ultraviolet (UV) light, gamma radiation, ethylene oxide, electron beams, and/or an autoclave.

Also disclosed herein are embodiments of a kit or tray including a first sinus implant having a first size, a second sinus implant having a second size that can be different than the first size, and a third sinus implant having a third size that can be different than the first and second sizes. In some embodiments, the first sinus implant can have a main body portion and a second body portion coupled with the main body portion, the second body portion extending at an angle away from the main body portion. In some embodiments, the first implant, the second implant, and the third implant can each be sized and configured for implantation in the frontal sinus. In some embodiments, a shape of the first implant, the second implant, and the third implant can each be similar to a shape of the frontal sinus of a patient that the first, second, or third implant will be implanted into. In some embodiments, the first, second, and third implants can have a similar shape. In some embodiments, the first, second, and third implants can each have a width from an anterior to a posterior surface of the implant that can be 15 mm or approximately 15 mm, 18 mm or approximately 18 mm, and 20 mm or approximately 20 mm, respectively. In some embodiments, the first, second, and third implants can each have a width from an anterior to a posterior surface of the implant that can be between about 15 mm and about 20 mm. In some embodiments, the first, second, and third implants can each have a length that can be 40 mm or approximately 40 mm, 55 mm or approximately 55 mm, and 70 mm or approximately 70 mm, respectively. In some embodiments, the first, second, and third implants can each have a length that is between about 40 mm and about 70 mm. In some embodiments, the first, second, and third implants can each have a height that can be 15 mm or approximately 15 mm, 25 mm or approximately 25 mm, and 30 mm or approximately 30 mm, respectively. In some embodiments, the first, second, and third implants can each have a height that is between about 15 mm and about 30 mm.

Disclosed herein are embodiments of a novel sinus implant device to assist surgeons with rapid frontal sinus repair or obliteration. Some embodiments of the sinus implant can be prefabricated to fit the morphology of most adult frontal sinuses based on 3D volumetric morphometric analysis and common shape and volume parameters.

Also disclosed herein are embodiments of a method of implanting a sinus implant device, which can be any embodiments of the novel implant device disclosed herein.

Benefits include an optimal fit to obliterate the potential space of the sinus and with reduced risk of CSF leak and infection, faster repair time and reduction of operative surgical and anesthesia time, drug eluting properties and reduced need for autologous grafting.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawing, may be arranged, substituted, combined, and designed in a wide variety of different configurations, all of which are explicitly contemplated and make part of this disclosure.

FIGS. 1A-1F show an embodiment of a sinus implant 100.

FIGS. 2A-2H show another embodiment of a sinus implant 200.

FIGS. 3A-3F show another embodiment of a sinus implant 200.

FIG. 4 shows a comparison of three different sized sinus implant embodiments.

### DETAILED DESCRIPTION OF SOME EXEMPLIFYING EMBODIMENTS

Disclosed herein are embodiments of a device for treating a sinus cavity or condition.

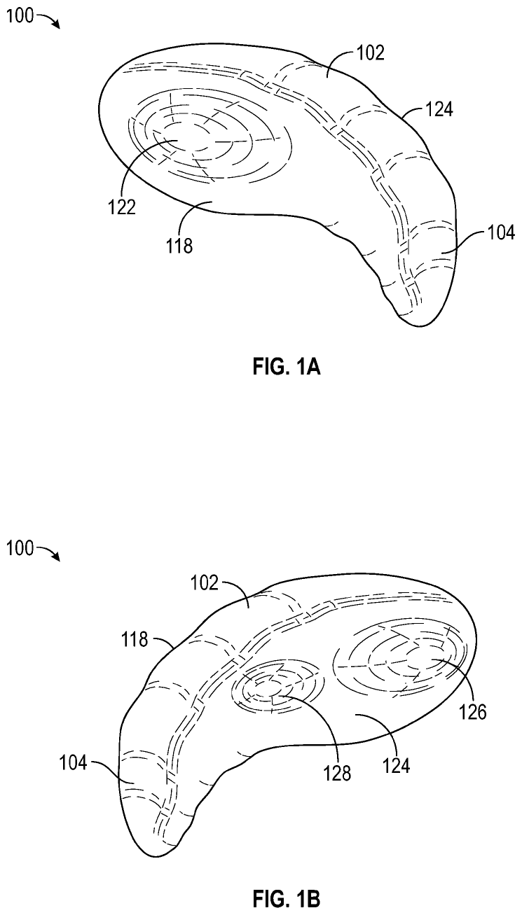

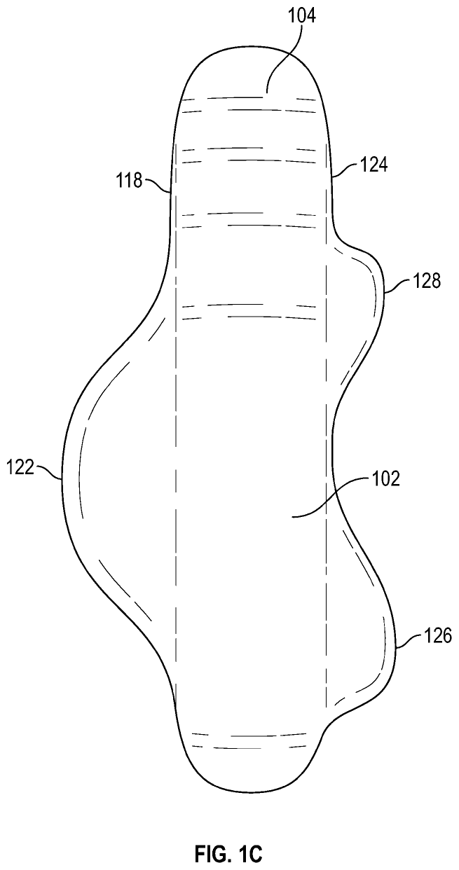

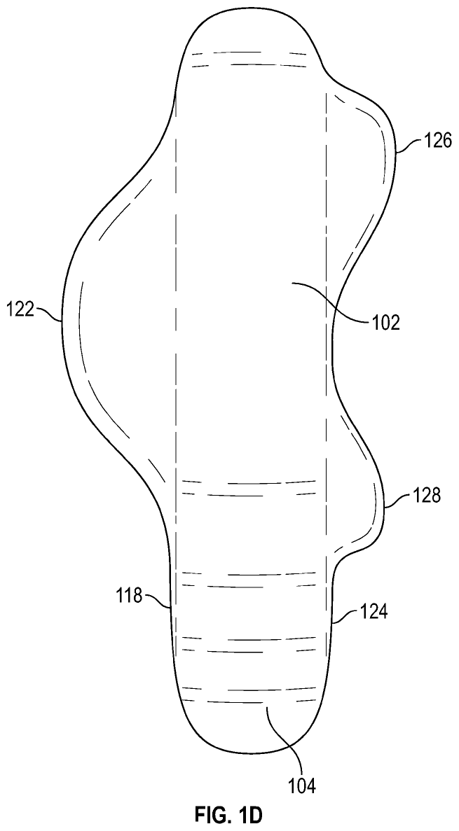

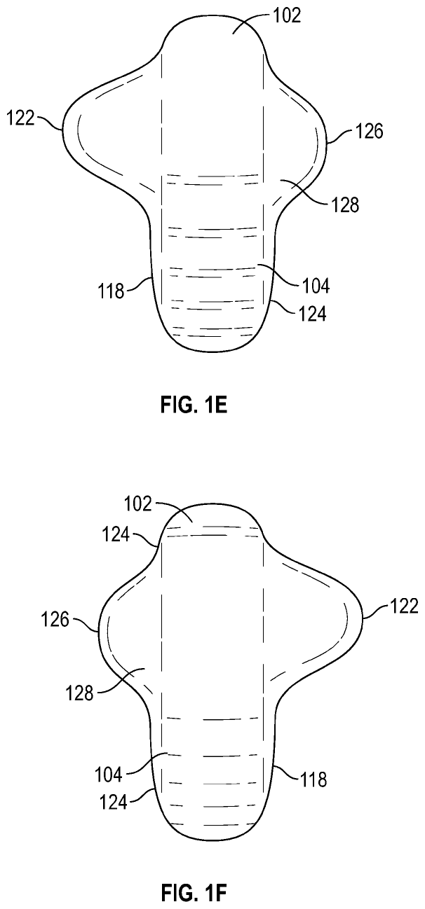

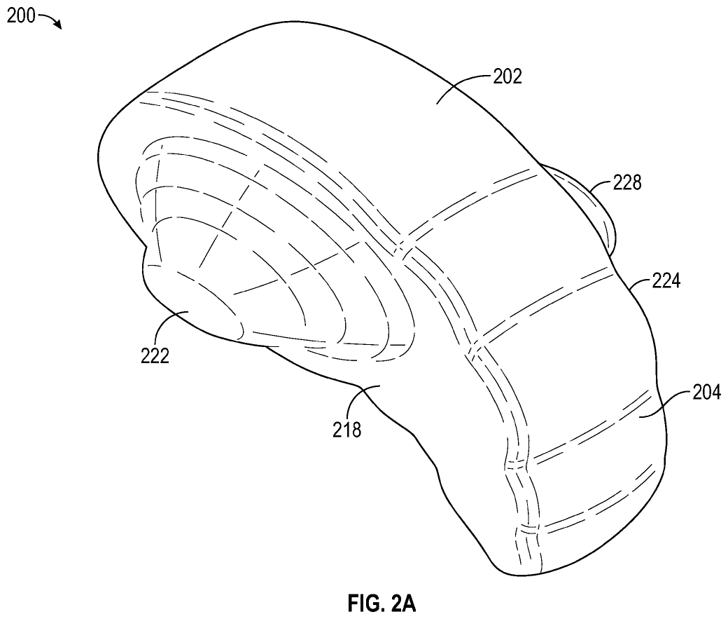

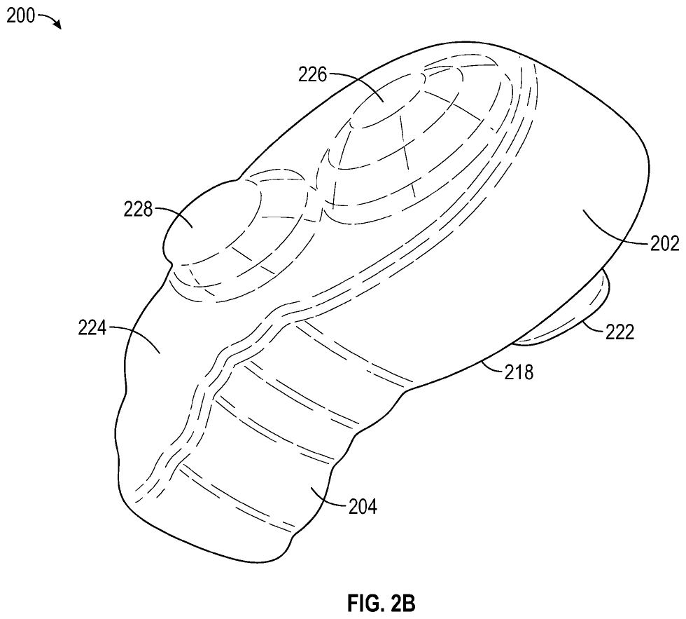

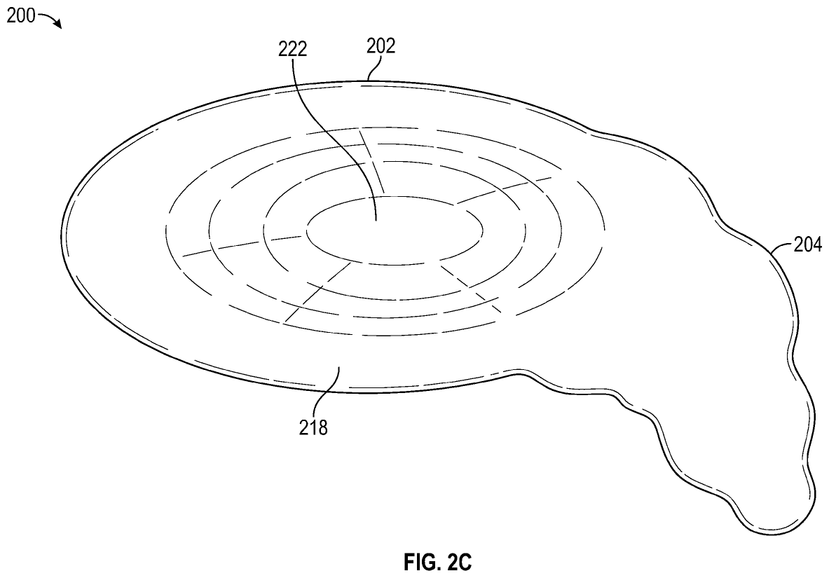

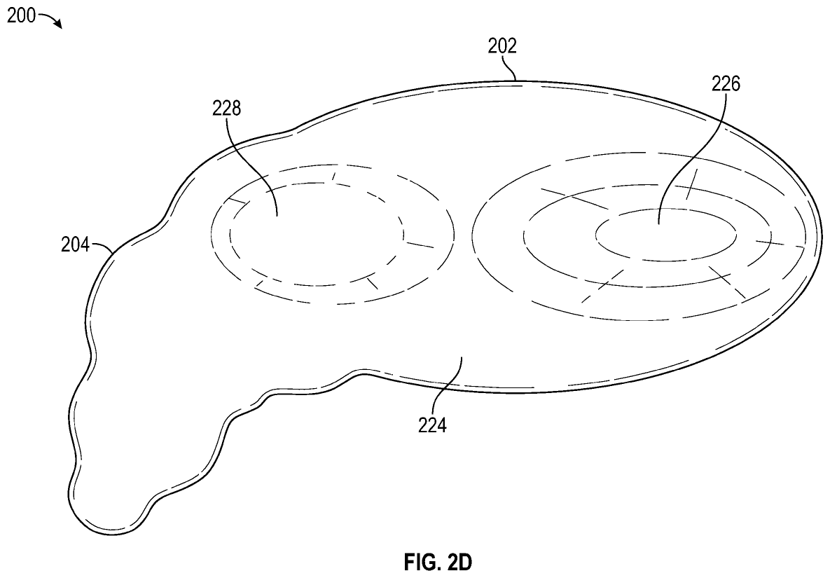

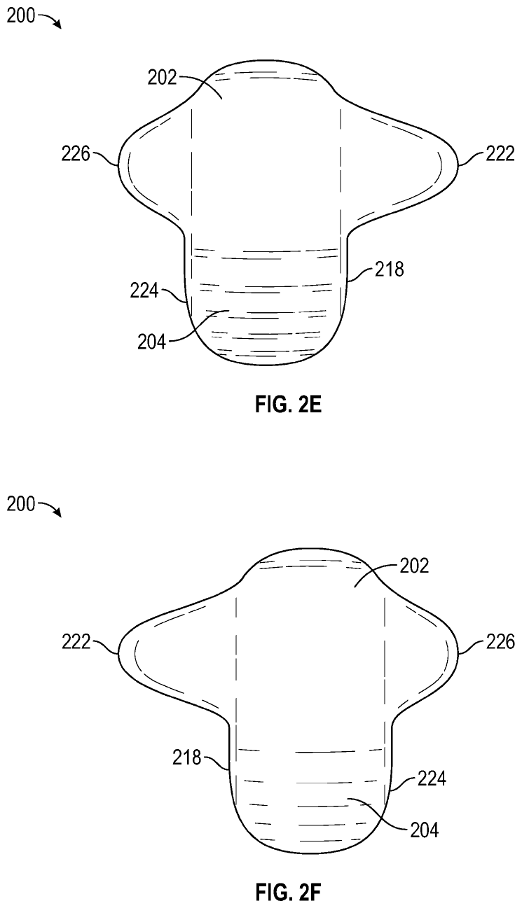

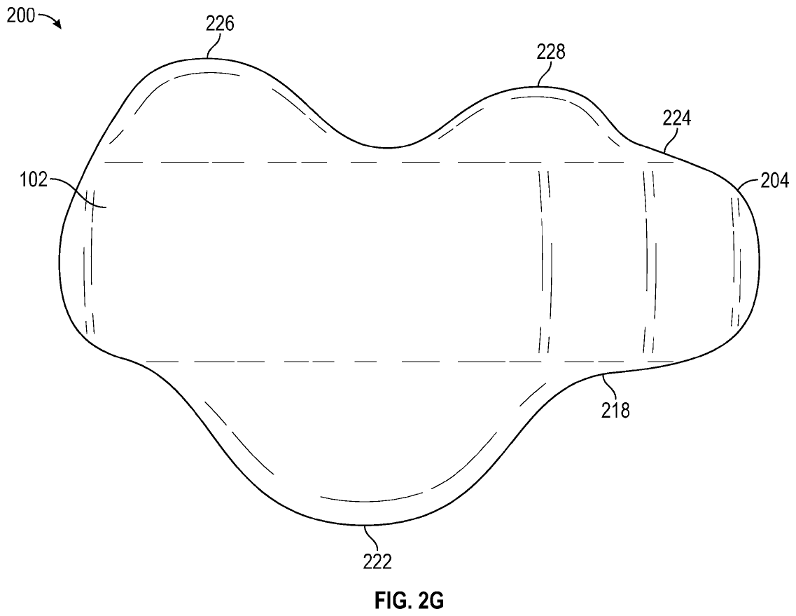

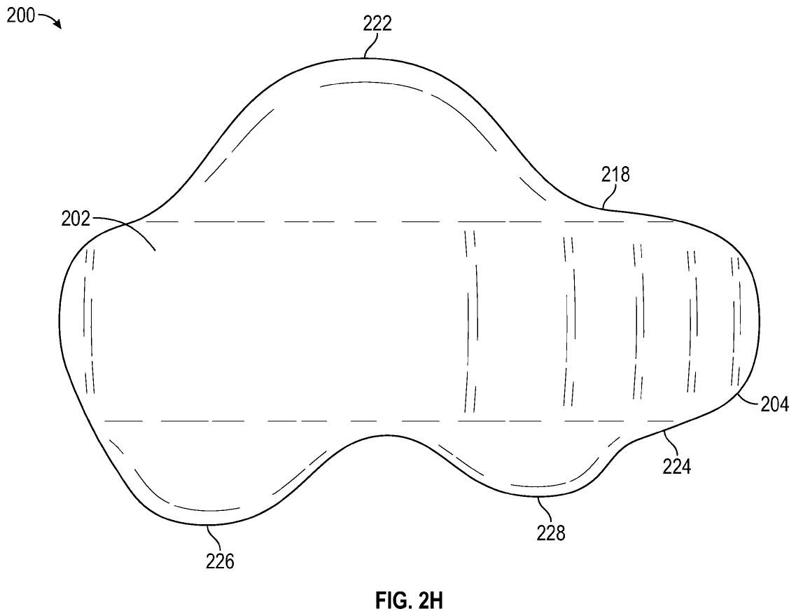

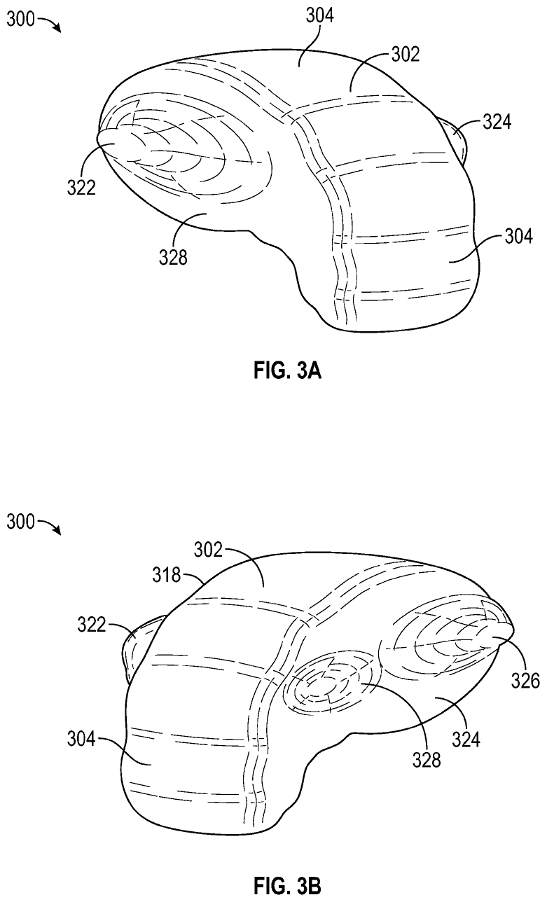

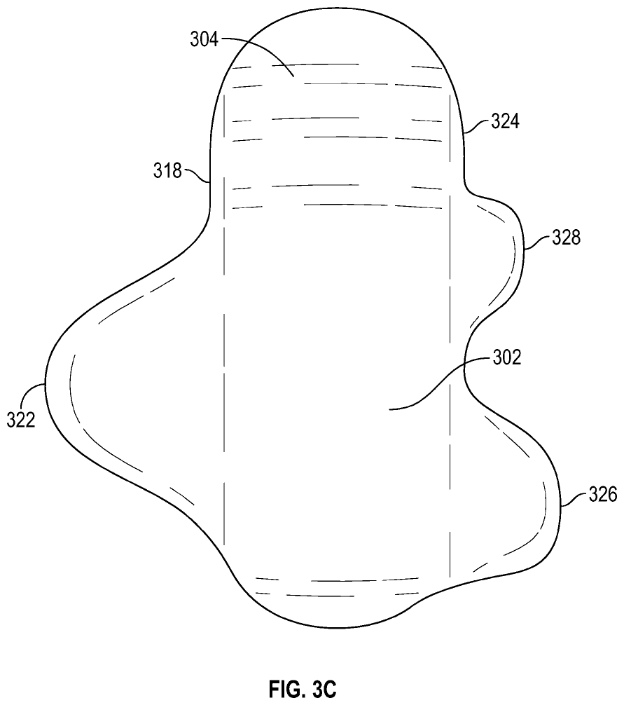

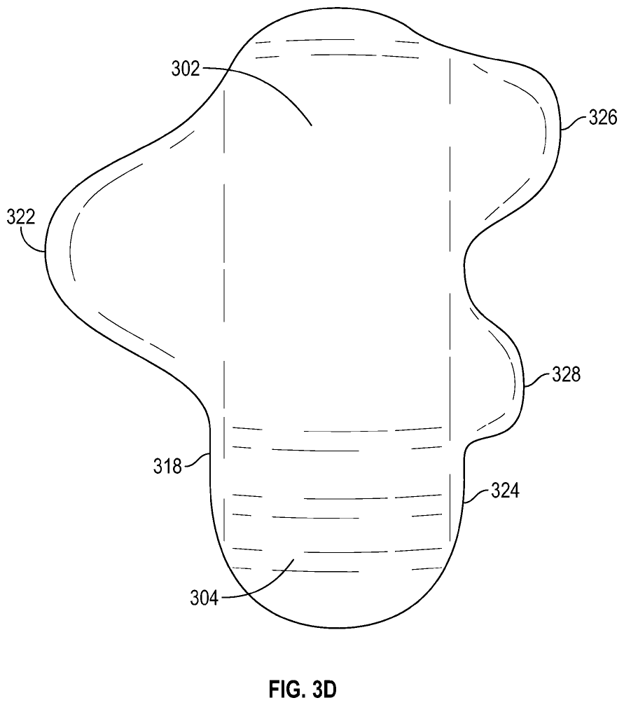

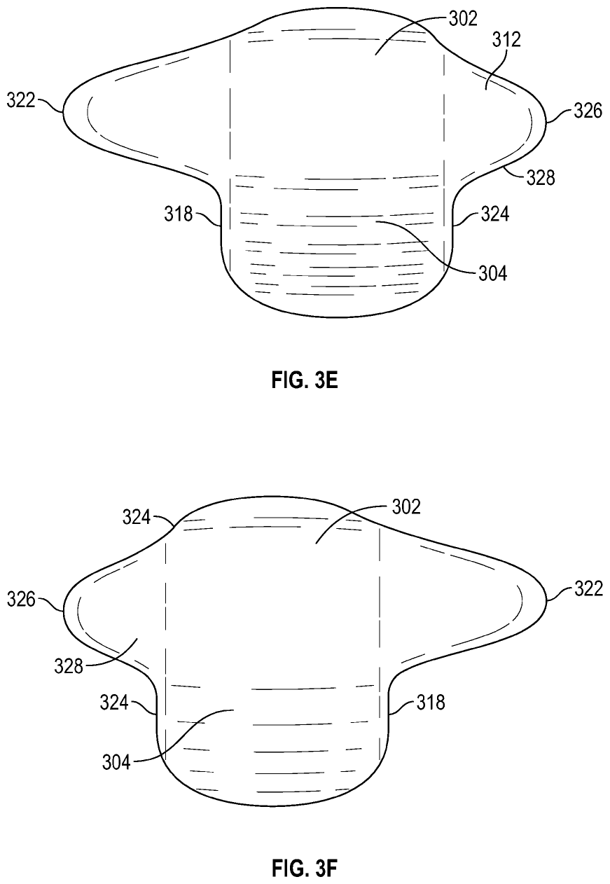

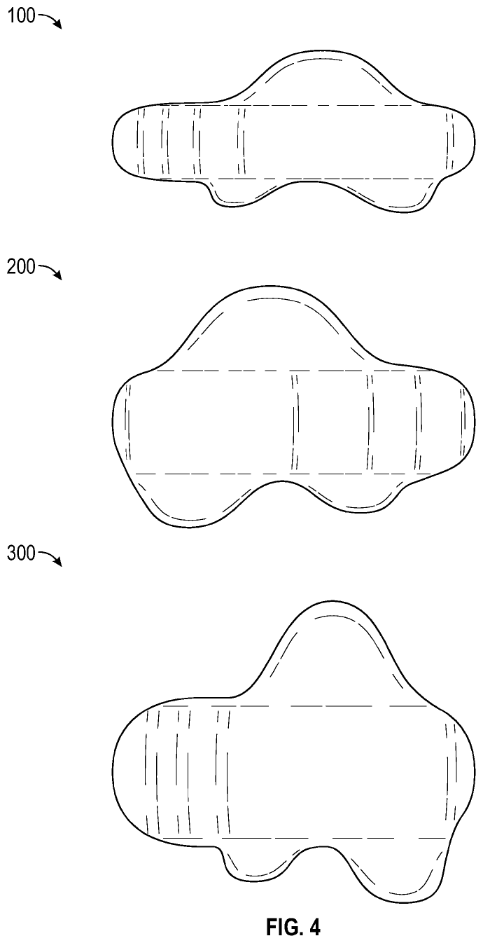

Though not so limited, FIGS. 1A-3F and FIG. 4 depict three example embodiments of a sinus implant 100, 200, and 300, showing three possible sizes and embodiments of a sinus implant. These sample embodiments illustrate that in some embodiments, the implant could come in three predetermined sizes and/or arrangements. FIGS. 1A-1F depict the smallest size of the three sample embodiments. FIGS. 2A-2G depict an intermediate size of a sample embodiment. FIGS. 3A-3F depict the largest of the three illustrative sample embodiments. Note that, in any embodiments disclosed herein, the implants can have smooth outside surface, or at least a higher resolution surface contour than shown in the figures. In some embodiments, a ratio of a length of some embodiments of the sinus implant 100 to a length of some embodiments of the sinus implant 200 can be 1:3 or approximately 1:3, as shown in FIG. 4. In some embodiments, a ratio of a length of some embodiments of the sinus implant 100 to a length of some embodiments of the sinus implant 300 can be 1:5 or approximately 1:5, as shown in FIG. 4.

As mentioned, FIGS. 1A-1F show a first embodiment of a sinus implant 100. In some embodiments, the sinus implant 100 can include a main body portion 102 (also referred to herein as a main body or a first portion) and a second body portion 104 (also referred to herein as an adjunct portion). In some embodiments, the main body portion 102 of the sinus implant 100 can be sized and shaped to approximately match a main portion of a patient's frontal sinus, or can be sized and shaped to approximately match a main portion of a range of patients'frontal sinus. Similarly, in some embodiments, a second portion 104 of the sinus implant 100 can be sized and shaped to approximately match a second portion of a patient's frontal sinus, or can be sized and shaped to approximately match a second portion of a range of patients'frontal sinus. For example, the second portion 104 of the sinus implant 100 can be sized and shaped to approximately match the frontal sinus infundibulum and/or the frontal recess of a patient's frontal sinus, or a range of patients'frontal sinus. In some embodiments, the sinus implant 100 can be curved along a length of the sinus implant 100. As will be described in more detail below, for some embodiments, a size and a shape of the sinus implant 100 is based on image data from a wide range of patients so that the sinus implant 100 approximately matches a size and a shape of the frontal sinus of a range of patients, or at least a portion of the size and the shape of the frontal sinus of a range of patients.

In some embodiments, the main body portion can be approximately elliptically shaped. In some embodiments, a shape of the sinus implant 100 can be constructed from dimensions taken from a 3D volumetric morphometric analysis of an individual. If designed based on an individual, the analysis can be completed and reviewed before the operation or can be completed and reviewed in the operating room. Alternatively, the shape of the sinus implant can be based off a combination of 3D volumetric analyses of multiple individuals to determine a common morphology or morphologies of the sinus. Alternatively, the implant may be moldable, trimmable, and cuttable intraoperatively by the surgeon to provide a best fit and to mold and plug or obliterate the sinus and outflow duct more effectively. Alternatively, the shape can be based on the amount of bony material removed during the procedure. For example, the implant can be custom printed and shaped to match any aberrations or cavities in the sinus cavity. In some embodiments, the sinus implant 100 can have anterior protrusion 122. Any embodiments of any implant disclosed herein can have one, two, or more anterior protrusions and/or one or more posterior protrusions. There can be one, two, or more posterior protrusions. The protrusions can be the same or of similar size or shape or of different sizes and/or shapes. In some embodiments, any of the protrusions can have a conical type shape, or a truncated conical type shape. An angle or slope of the surface of the protrusions can vary around the protrusions.

The protrusions can assist in orienting the sinus implant 100 within the sinus cavity. The tapered second body portion of some embodiments can have the functionality of fitting within the sinus cavity, specifically filling the tapered section that allows for fluids to run into the sinus recess. The tapered second body portion of some embodiments can extend from the main body portion so that the tapered second body portion is positioned in the tapered section of the sinus cavity. For example, the tapered second body portion can fit within the frontal sinus infundibulum and/or the frontal recess. This can have benefits over current methods such as using fat to fill the sinus recess, as a craniotomy may not expose the sinus recess and therefore fat cannot be manually inserted into the sinus recess.

Some embodiments of the implant 100 can have a length of 40 mm or approximately 40 mm, a width of 15 mm or approximately 15 mm, and a height of 15 mm or approximately 15 mm, or less or more than these values.

FIGS. 1D-1F depict top, bottom, front, and rear views respectively of the embodiment of the sinus implant 100 shown in FIG. 1A. In some embodiments, the anterior protrusion on anterior face can be of a longer length than the posterior protrusions on posterior face. In other embodiments, the posterior protrusions can be longer than anterior protrusion. Additionally, the posterior protrusions can be of different lengths. In some other embodiments, the posterior protrusions can be of the same length. The width of the main body can be the same as the tapered adjunction. In some embodiments, the widths of the main body and the tapered adjunction can be different.

The sinus implant 100 of some embodiments can be formed in a plurality of different sizes and/or shapes configured to approximately match a size and/or a shape of a wide range of patients. For example and without limitation, in some embodiments, the sinus implant 100 can be formed in a first size and shape for a first range or group of patients, and/or a second size and shape for a second range or group of patients, wherein the second size is larger than the first size, and/or a third size and shape for a third range or group of patients, wherein the third size is larger than the second size. In some embodiments, the sinus implant 100 can be formed in any number of different sizes, including more than three different sizes.

### Anterior Surface, Posterior Surface, Protrusions

Some embodiments of the sinus implant 100 can have an anterior surface 118 and a posterior surface 124. In some embodiments, the anterior surface 118 and/or the posterior surface 124 can be approximately planar. In some embodiments, the anterior surface 118 and/or the posterior surface 124 can be curved or rounded. In some embodiments, the anterior surface 118 and/or the posterior surface 124 can be smooth, for example and without limitation, versus step-like in portions as shown in the figures. In some embodiments, the anterior surface 118 and/or the posterior surface 124 can be stepped or tiered. In some embodiments, the posterior surface 124 can be approximately planar. In some embodiments, the posterior surface 124 can be curved or rounded.

In some embodiments, the main body portion 102 can have an anterior surface 118 that can have an anterior protrusion 122 extending away from the anterior surface 118, or two or more anterior protrusions 122 extending away from the anterior surface 118. In some embodiments, anterior protrusions 122 can extend away from the anterior surface 118 at an angle that is approximately perpendicular to the anterior surface 118. The anterior protrusion 122 can have a tapered shape wherein a base of the anterior protrusion 122 is wider and longer than a distal end portion of the anterior protrusion 122. In some embodiments, the anterior protrusion 122 can have an approximately conical shape. In some embodiments, the anterior protrusion 122 can be configured to fill or partially fill a recess or cavity in the frontal sinus. The anterior protrusion 122 can be sized and shaped to fill a corresponding recess or feature of a range of patients. For example and without limitation, the anterior protrusion 122 can have a size that matches or approximately matches median or average size and shape of a corresponding recess or cavity in the frontal sinus of a range of patients. Some embodiments of the sinus implant 100 do not have any protrusions extending from the anterior surface 118.

A posterior surface 124 of the main body portion 102 can also have one or more or two or more protrusions extending away from the posterior surface 124. For example and without limitation, some embodiments of the sinus implant 100 can have a first and a second posterior protrusion 126, 128 extending away from the posterior surface 124. In some embodiments, the first posterior protrusion 126 can extend away from the posterior surface 124 at an angle that is approximately perpendicular to the posterior surface 124.

In some embodiments, an outer surface of anterior protrusion or protrusions 122 and/or the posterior protrusions 126, 128 can be smooth. In some embodiments, an outer surface of anterior and posterior protrusions 122, 126, and/or the second posterior protrusion 128 can be stepped or tiered. For example and without limitation, some embodiments of the protrusions 122, 126, and/or the second posterior protrusion 128 can have three or more different tiers or levels from a proximal end thereof to a distal end thereof, or four or more different tiers or levels from a proximal end thereof to a distal end thereof, or five or more different tiers or levels from a proximal end thereof to a distal end thereof.

Either or both of the posterior protrusions 126, 128 can have a tapered shape wherein a base of each posterior protrusion 126, 128 is wider and longer than a distal end portion of the posterior protrusions 126, 128. In some embodiments, the posterior protrusions 126, 128 can each have an approximately conical shape. In some embodiments, the posterior protrusions 126, 128 can be configured to fill or partially fill recesses or cavities in the frontal sinus. The posterior protrusions 126, 128 can be sized and shaped to fill a corresponding recess or feature of a range of patients. For example and without limitation, the posterior protrusions 126, 128 can each have a size that matches or approximately matches median or average size and shape of a corresponding recess or cavity in the frontal sinus of a range of patients. Some embodiments of the sinus implant 100 do not have any protrusions extending from the posterior surface 124.

In some embodiments, the anterior protrusion 122, the first posterior protrusion 126, and/or the second posterior protrusion 128 can be configured to fill a recess or cavity in the frontal sinus, and can be sized and shaped according to a median or average size and shape or a range of sizes and shapes of the recesses. Some embodiments of the sinus implant 100 have only one protrusion extending away from the posterior surface 124. Some embodiments of the sinus implant 100 do not have any protrusions extending from the posterior surface 124.

FIGS. 2A-2H depict another embodiment of a sinus implant 200. In any embodiments disclosed herein, the sinus implant 200 can have any of the components, features, or other details of any other sinus implant embodiments disclosed herein, including without limitation, embodiments of the sinus implant 100 described herein, in any combination with any of the components, features, or other details of the sinus implant 200 shown in FIGS. 2A-2H and described herein. Similarly, any components, features, or other details of any of the other sinus implants disclosed herein, including the sinus implant 100, can have any of the components, features, or other details of any embodiments of the sinus implant 200.

In some embodiments, the sinus implant 200 can be larger than some embodiments of the sinus implant 100. As with some embodiments of the implant 100, some embodiments of the implant 200 can have a main body portion 202 (also referred to herein as a main body or a first portion) and a second body portion 204 (also referred to herein as an adjunct portion). In some embodiments, the main body portion 202 of the implant 200 can be longer, wider, and/or taller than the main body portion 102 of the sinus implant 100. In some embodiments, the entire implant 200 can be longer, wider, and/or taller than the entire sinus implant 100. In some embodiments, the main body portion 202 of the sinus implant 200 can be sized and shaped to approximately match a main portion of a patient's frontal sinus, or can be sized and shaped to approximately match a main portion of a range of patients'frontal sinus. Similarly, in some embodiments, a second portion 204 of the sinus implant 200 can be sized and shaped to approximately match a second portion of a patient's frontal sinus, or can be sized and shaped to approximately match a second portion of a range of patients'frontal sinus. For example, the second portion 204 of the sinus implant 200 can be sized and shaped to approximately match the frontal sinus infundibulum and/or the frontal recess of a patient's frontal sinus, or a range of patients'frontal sinus. In some embodiments, the sinus implant 100 can be curved along a length of the sinus implant 100. As will be described in more detail below, for some embodiments, a size and a shape of the sinus implant 200 is based on image data from a wide range of patients so that the sinus implant 200 approximately matches a size and a shape of the frontal sinus of a range of patients, or at least a portion of the size and the shape of the frontal sinus of a range of patients.

In some embodiments, the main body portion can be approximately elliptically shaped. In some embodiments, a shape of the sinus implant 200 can be constructed from dimensions taken from a 3D volumetric morphometric analysis of an individual. If designed based on an individual, the analysis can be completed and reviewed before the operation or can be completed and reviewed in the operating room. Alternatively, the shape of the sinus implant can be based off a combination of 3D volumetric analyses of multiple individuals to determine a common morphology of the sinus. Alternatively, the shape can be based on the amount of bony material removed during the procedure. In some embodiments, the sinus implant 200 can have anterior protrusion 222. There can be one, two, or more anterior protrusions and/or one or more posterior protrusions. There can be one, two, or more posterior protrusions. The protrusions can be the same or of similar size/shape or of different sizes/shapes.

Some embodiments of the implant 200 can have a length of 55 mm or approximately 55 mm, a width of 18 mm or approximately 18 mm, and a height of 25 mm or approximately 25 mm, or less or more than these values. The protrusions can assist in orienting the sinus implant 200 within the sinus cavity. The tapered second body portion of some embodiments can have the functionality of fitting within the sinus cavity, specifically filling the tapered section that allows for fluids to run into the sinus recess. The tapered second body portion of some embodiments can extend from the main body portion so the tapered second body portion is positioned in the tapered section of the sinus cavity. For example, the tapered second body portion can fit within the frontal sinus infundibulum and/or the frontal recess. This can have benefits over current methods such as using fat to fill the sinus recess, as a craniotomy may not expose the sinus recess and therefore fat cannot be manually inserted into the sinus recess.

In some embodiments, the anterior protrusion on anterior face can be of a longer length than the posterior protrusions on posterior face. In other embodiments, the posterior protrusions can be longer than anterior protrusion. Additionally, the posterior protrusions can be of different lengths. In some other embodiments, the posterior protrusions can be of the same length. The width of the main body can be the same as the tapered adjunction. In some embodiments, the widths of the main body and the tapered adjunction can be different.

The sinus implant 200 of some embodiments can be formed in a plurality of different sizes and/or shapes configured to approximately match a size and/or a shape of a range of patients. For example and without limitation, in some embodiments, the sinus implant 200 can be formed in a first size and shape for a first range or group of patients, and/or a second size and shape for a second range or group of patients, wherein the second size is larger than the first size, and/or a third size and shape for a third range or group of patients, wherein the third size is larger than the second size. In some embodiments, the sinus implant 200 can be formed in any number of different sizes, including more than three different sizes.

### Anterior Surface, Posterior Surface, Protrusions

Some embodiments of the sinus implant 200 can have an anterior surface 218 and a posterior surface 224. In some embodiments, the anterior surface 218 and/or the posterior surface 224 can be approximately planar. In some embodiments, the anterior surface 218 and/or the posterior surface 224 can be curved or rounded. In some embodiments, the anterior surface 218 and/or the posterior surface 224 and any projections thereon, including without limitation projections 224, 226, 228 can be smooth. In some embodiments, the anterior surface 218 and/or the posterior surface 224 and any protrusions or features thereon can be stepped or tiered. In some embodiments, the posterior surface 224 can be approximately planar. In some embodiments, the posterior surface 224 can be curved or rounded.

In some embodiments, the main body portion 202 can have an anterior surface 218 that can have an anterior protrusion 222 extending away from the anterior surface 218, or two or more anterior protrusions 222 extending away from the anterior surface 218. In some embodiments, anterior protrusions 222 can extend away from the anterior surface 218 at an angle that is approximately perpendicular to the anterior surface 218. The anterior protrusion 222 can have a tapered shape wherein a base of the anterior protrusion 222 is wider and longer than a distal end portion of the anterior protrusion 222. In some embodiments, the anterior protrusion 222 can have an approximately conical shape. In some embodiments, the anterior protrusion 222 can be configured to fill or partially fill a recess or cavity in the frontal sinus. The anterior protrusion 222 can be sized and shaped to fill a corresponding recess or feature of a range of patients. For example and without limitation, the anterior protrusion 222 can have a size that matches or approximately matches median or average size and shape of a corresponding recess or cavity in the frontal sinus of a range of patients. Some embodiments of the sinus implant 200 do not have any protrusions extending from the anterior surface 218.

A posterior surface 224 of the main body portion 202 can also have one or more or two or more protrusions extending away from the posterior surface 224. For example and without limitation, some embodiments of the sinus implant 200 can have a first and a second posterior protrusion 226, 228 extending away from the posterior surface 224. In some embodiments, the first posterior protrusion 226 can extend away from the posterior surface 224 at an angle that is approximately perpendicular to the posterior surface 224.

In some embodiments, an outer surface of anterior protrusion or protrusions 222 and/or the posterior protrusions 226, 228 can be smooth. In some embodiments, an outer surface of anterior and posterior protrusions 222, 226, and/or the second posterior protrusion 228 can be stepped or tiered. For example and without limitation, some embodiments of the protrusions 222, 226, and/or the second posterior protrusion 228 can have three or more different tiers or levels from a proximal end thereof to a distal end thereof, or four or more different tiers or levels from a proximal end thereof to a distal end thereof, or five or more different tiers or levels from a proximal end thereof to a distal end thereof.

Either or both of the posterior protrusions 226, 228 can have a tapered shape wherein a base of each posterior protrusion 226, 228 is wider and longer than a distal end portion of the posterior protrusions 226, 228. In some embodiments, the posterior protrusions 226, 228 can each have an approximately conical shape. In some embodiments, the posterior protrusions 226, 228 can be configured to fill or partially fill recesses or cavities in the frontal sinus. The posterior protrusions 226, 228 can be sized and shaped to fill a corresponding recess or feature of a range of patients. For example and without limitation, the posterior protrusions 226, 228 can each have a size that matches or approximately matches median or average size and shape of a corresponding recess or cavity in the frontal sinus of a range of patients. Some embodiments of the sinus implant 200 do not have any protrusions extending from the posterior surface 224.

In some embodiments, the anterior protrusion 222, the first posterior protrusion 226, and/or the second posterior protrusion 228 can be configured to fill a recess or cavity in the frontal sinus, and can be sized and shaped according to a median or average size and shape or a range of sizes and shapes of the recesses. Some embodiments of the sinus implant 200 have only one protrusion extending away from the posterior surface 224. Some embodiments of the sinus implant 200 do not have any protrusions extending from the posterior surface 224.

FIGS. 3A-3F depict another embodiment of a sinus implant 300. In any embodiments disclosed herein, the sinus implant 300 can have any of the components, features, or other details of any other sinus implant embodiments disclosed herein, including without limitation, embodiments of the sinus implant 100, 200 described herein, in any combination with any of the components, features, or other details of the sinus implant 300 shown in FIGS. 3A-3F and/or described herein. Similarly, any components, features, or other details of any of the other sinus implants disclosed herein, including the sinus implant 100, 200, can have any of the components, features, or other details of any embodiments of the sinus implant 300.

In some embodiments, the sinus implant 300 can be larger than some embodiments of the sinus implant 200. As with some embodiments of the implant 100, some embodiments of the implant 300 can have a main body portion 302 (also referred to herein as a main body or a first portion) and a second body portion 304 (also referred to herein as an adjunct portion). In some embodiments, the main body portion 302 of the implant 300 can be longer, wider, and/or taller than the main body portion 202 of the sinus implant 200. In some embodiments, the entire implant 300 can be longer, wider, and/or taller than the entire sinus implant 200. In some embodiments, the main body portion 302 of the sinus implant 300 can be sized and shaped to approximately match a main portion of a patient's frontal sinus, or can be sized and shaped to approximately match a main portion of a range of patients'frontal sinus. Similarly, in some embodiments, a second portion 304 of the sinus implant 300 can be sized and shaped to approximately match a second portion of a patient's frontal sinus, or can be sized and shaped to approximately match a second portion of a range of patients'frontal sinus. For example, the second portion 304 of the sinus implant 300 can be sized and shaped to approximately match the frontal sinus infundibulum and/or the frontal recess of a patient's frontal sinus, or a range of patients'frontal sinus. In some embodiments, the sinus implant 100 can be curved along a length of the sinus implant 300. As will be described in more detail below, for some embodiments, a size and a shape of the sinus implant 300 is based on image data from a wide range of patients so that the sinus implant 300 approximately matches a size and a shape of the frontal sinus of a range of patients, or at least a portion of the size and the shape of the frontal sinus of a range of patients.

In some embodiments, the main body portion can be approximately elliptically shaped. In some embodiments, a shape of the sinus implant 300 can be constructed from dimensions taken from a 3D volumetric morphometric analysis of an individual. If designed based on an individual, the analysis can be completed and reviewed before the operation or can be completed and reviewed in the operating room. Alternatively, the shape of the sinus implant can be based off a combination of 3D volumetric analyses of multiple individuals to determine a common morphology of the sinus. Alternatively, the shape can be based on the amount of bony material removed during the procedure. In some embodiments, the sinus implant 300 can have anterior protrusion 322. There can be one, two, or more anterior protrusions and/or one or more posterior protrusions. There can be one, two, or more posterior protrusions. The protrusions can be the same or of similar size/shape or of different sizes/shapes.

Some embodiments of the implant 300 can have a length of 70 mm or approximately 70 mm, a width of 20 mm or approximately 20 mm, and a height of 30 mm or approximately 30 mm, or less or more than these values. The protrusions can assist in orienting the sinus implant 200 within the sinus cavity. The protrusions can assist in orienting the sinus implant 300 within the sinus cavity. The tapered second body portion of some embodiments can have the functionality of fitting within the sinus cavity, specifically filling the tapered section that allows for fluids to run into the sinus recess. The tapered second body portion of some embodiments can extend from the main body portion so the tapered second body portion is positioned in the tapered section of the sinus cavity. For example, the tapered second body portion can fit within the frontal sinus infundibulum and/or the frontal recess. This can have benefits over current methods such as using fat to fill the sinus recess, as a craniotomy may not expose the sinus recess and therefore fat cannot be manually inserted into the sinus recess.

In some embodiments, the anterior protrusion on anterior face can be of a longer length than the posterior protrusions on posterior face. In other embodiments, the posterior protrusions can be longer than anterior protrusion. Additionally, the posterior protrusions can be of different lengths. In some other embodiments, the posterior protrusions can be of the same length. The width of the main body can be the same as the tapered adjunction. In some embodiments, the widths of the main body and the tapered adjunction can be different.

The sinus implant 300 of some embodiments can be formed in a plurality of different sizes and/or shapes configured to approximately match a size and/or a shape of a range of patients. For example and without limitation, in some embodiments, the sinus implant 300 can be formed in a first size and shape for a first range or group of patients, and/or a second size and shape for a second range or group of patients, wherein the second size is larger than the first size, and/or a third size and shape for a third range or group of patients, wherein the third size is larger than the second size. In some embodiments, the sinus implant 300 can be formed in any number of different sizes, including more than three different sizes.

### Anterior Surface, Posterior Surface, Protrusions

Some embodiments of the sinus implant 300 can have an anterior surface 318 and a posterior surface 324. In some embodiments, the anterior surface 318 and/or the posterior surface 324 can be approximately planar. In some embodiments, the anterior surface 318 and/or the posterior surface 324 can be curved or rounded. In some embodiments, the anterior surface 318 and/or the posterior surface 324 can be smooth. In some embodiments, the anterior surface 318 and/or the posterior surface 324 can be stepped or tiered. In some embodiments, the posterior surface 324 can be approximately planar. In some embodiments, the posterior surface 324 can be curved or rounded.

In some embodiments, the main body portion 302 can have an anterior surface 318 that can have an anterior protrusion 322 extending away from the anterior surface 318, or two or more anterior protrusions 322 extending away from the anterior surface 318. In some embodiments, anterior protrusions 322 can extend away from the anterior surface 318 at an angle that is approximately perpendicular to the anterior surface 318. The anterior protrusion 322 can have a tapered shape wherein a base of the anterior protrusion 322 is wider and longer than a distal end portion of the anterior protrusion 322. In some embodiments, the anterior protrusion 322 can have an approximately conical shape. In some embodiments, the anterior protrusion 322 can be configured to fill or partially fill a recess or cavity in the frontal sinus. The anterior protrusion 322 can be sized and shaped to fill a corresponding recess or feature of a range of patients. For example and without limitation, the anterior protrusion 322 can have a size that matches or approximately matches median or average size and shape of a corresponding recess or cavity in the frontal sinus of a range of patients. Some embodiments of the sinus implant 300 do not have any protrusions extending from the anterior surface 318.

A posterior surface 324 of the main body portion 302 can also have one or more or two or more protrusions extending away from the posterior surface 324. For example and without limitation, some embodiments of the sinus implant 300 can have a first and a second posterior protrusion 326, 328 extending away from the posterior surface 324. In some embodiments, the first posterior protrusion 326 can extend away from the posterior surface 324 at an angle that is approximately perpendicular to the posterior surface 324.

In some embodiments, an outer surface of anterior protrusion or protrusions 322 and/or the posterior protrusions 326, 328 can be smooth. In some embodiments, an outer surface of anterior and posterior protrusions 322, 326, and/or the second posterior protrusion 328 can be stepped or tiered. For example and without limitation, some embodiments of the protrusions 322, 326, and/or the second posterior protrusion 328 can have three or more different tiers or levels from a proximal end thereof to a distal end thereof, or four or more different tiers or levels from a proximal end thereof to a distal end thereof, or five or more different tiers or levels from a proximal end thereof to a distal end thereof.

Either or both of the posterior protrusions 326, 328 can have a tapered shape wherein a base of each posterior protrusion 326, 328 is wider and longer than a distal end portion of the posterior protrusions 326, 328. In some embodiments, the posterior protrusions 326, 328 can each have an approximately conical shape. In some embodiments, the posterior protrusions 326, 328 can be configured to fill or partially fill recesses or cavities in the frontal sinus. The posterior protrusions 326, 328 can be sized and shaped to fill a corresponding recess or feature of a range of patients. For example and without limitation, the posterior protrusions 326, 328 can each have a size that matches or approximately matches median or average size and shape of a corresponding recess or cavity in the frontal sinus of a range of patients. Some embodiments of the sinus implant 300 do not have any protrusions extending from the posterior surface 324.

In some embodiments, the anterior protrusion 322, the first posterior protrusion 326, and/or the second posterior protrusion 328 can be configured to fill a recess or cavity in the frontal sinus, and can be sized and shaped according to a median or average size and shape or a range of sizes and shapes of the recesses. Some embodiments of the sinus implant 300 have only one protrusion extending away from the posterior surface 324. Some embodiments of the sinus implant 300 do not have any protrusions extending from the posterior surface 324.

In any embodiments disclosed herein, the main body portion 302, the second body portion 304, and/or any other portions or features of the device 300 (including any of the protrusions) can be sized and shaped to be similar to or approximately match a frontal sinus or one or more recesses or features of the frontal sinus of a patient. In some embodiments, a shape and/or a size the implant device 300 can be based on anatomical data gathered from a large number of CT scans and/or other imaging or measurement data of a frontal sinus portion of a range of people.

### Anatomical Data

In any embodiments disclosed herein, the main body portion 102, 202, 302 the second body portion 104, 204, 304 and/or any other portions or features of the implant 100, 200, 300 and/or any of the protrusions can be sized and shaped to be similar to or approximately match a frontal sinus or one or more recesses or features of the frontal sinus of a patient. In some embodiments, a shape and/or a size the implant device 100, 200, 300 can be based on anatomical data gathered from a large number of CT scans and/or other imaging or measurement data of a frontal sinus portion of a range of people.

### Material

Any embodiments of the sinus implant (including implant 100, 200, 300) can be composed of a core material and an outer material that can be different than the core material. For example and without limitation, the core material can be of a greater hardness than the outer material. In some embodiments, the inner material can be polyethylene (PE), high density polyethylene (HDPE), polymethyl methacrylate (PMMA), a polyester such as PLA, polyethylene glycol (PEG), polydimethylsiloxane (PDMS), or polydioxanone (PDS), or any other suitable material or any combination, blend, or copolymer of any of the foregoing materials. In some embodiments, the core material can be polyethylene (PE), high density polyethylene (HDPE), polymethyl methacrylate (PMMA), a polyester such as PLA, polyethylene glycol (PEG), polydimethylsiloxane (PDMS), or polydioxanone (PDS), or any other suitable material or any combination, blend, or copolymer of any of the foregoing materials. In some embodiments, the core material can be any thermoplastic polymer with any suitable or desired elastic modulus, hardness, and biocompatibility. The core material can include any combination of any of the foregoing and/or other suitable materials. Such materials can be designed to give the device better physical stability with elastic modulus of 500 MPa or more, in some embodiments. In any embodiments disclosed herein the elastic modulus of the implant can be from 5 to 500 MPa, or from approximately 5 to approximately 500 MPa, or of any values or ranges within these ranges. In any embodiments disclosed herein, the elastic modulus can be from5 or approximately 5 MPa to 500 or approximately 500 MPa. For example implants made from PDMS can have an elastic modulus of approximately 5 MPa. An implant made from a 3D printed plastic can have an elastic modulus of approximately 500 MPa.

The outer material of some embodiments of the sinus implant (including implant 100, 200, 300) can have any desired thickness. For example and without limitation, the outer material can have a thickness that is 10%, or approximately 10%, or 10% or less of the total thickness of the sinus implant (including implant 100, 200, 300), or 20%, or approximately 20%, or 20% or less of the total thickness of the sinus implant (including implant 100, 200, 300), or from 20% or approximately 20% to 50% or approximately 50% of the total thickness of the sinus implant 100, 200, 300.

In any embodiments, the sinus implant 100, 200, 300 can be made from a biocompatible material such that the sinus implant is biocompatible. In such embodiments, the sinus implant can be a permanent implant. Alternatively, some embodiments of the sinus implant 100 can be biodegradable or bioabsorbable such that the sinus implant 100 can gradually dissolve, for example and without limitation, over a period of five, seven, or ten days or less, or over a period of one month, two months, or six months or less, or over a period of one to three months, or one to six months, or one year or less, or two years or less, or five years or less. Having different products configured for different rates of bioabsorption provides the doctors with a greater freedom to prescribe the sinus implant that the doctor believes would be more beneficial for each patient. In some embodiments, the sinus implant 100, 200, 300 can be of such a material that the sinus implant can be stored at room temperature.

In some embodiments of the sinus implant 100, 200, 300, the outer material can be of lesser hardness relative to the core material. For example and without limitation, in some embodiments, the outer material can have an elastic modulus of 1 MPa or lower, or from 2-5 MPa, or 5 MPa or greater. In some embodiments, the outer material can be a thermoset crosslinked polymer. For example, in some embodiments, the outer material can include polydimethylsiloxane (PDMS), also referred to as dimethicone, polyethylene glycol (PEG), silicone, polydioxanone (PDS), or any other desired or suitable material or any combination of any of the foregoing materials such that the outer material can be a combination of multiple materials. In any embodiments, the sinus implant 100, 200, 300 can be homogenous such that there are no different layers of materials.

In some embodiments, the sinus implant 100, 200, 300 can be printed in different layers to have a gradient of hardness from core to outer surface. For example and without limitation, the hardness can decrease from the core to the outer surface such that the outer surface is softer or more pliable than the core. In some embodiments, the printed implant can be PEGylated for a better fit.

PEGylation is a common practice in biomedical devices that come in contact with body fluids. PEGylated devices can be bioinert and can reduce inflammation, and can also be charged with desired agents for drug delivery purposes. The implant of some embodiments can be dip-coated or sprayed on with polyethylene glycol (PEG) and its acrylate derivatives.

In some embodiments, a portion of the material of the sinus implant 100, 200, 300, such as without limitation an outer surface or outer layer portion of the sinus implant, can be infused or otherwise configured to include a substance, such as a therapeutic substance. In some embodiments, the substance can be an antibiotic, an anti-inflammatory, a steroid, or any other suitable or desired substance or combination of substances. Some embodiments of the sinus implant 100, 200, 300 can have multiple different substances added to the sinus implant 100, 200, 300, such as to the outer layer of the sinus implant. In some embodiments, the composition of the material can be such that the sinus implant 100, 200, 300 or at least an outer surface of the sinus implant is hydrophobic.

In some embodiments, the sinus implant 100, 200, 300 can be configured to expand within the sinus cavity after implantation of the sinus implant in the sinus cavity. For example and without limitation, some embodiments of the sinus implant 100, 200, 300 can be made from an expandable or expanding material or otherwise be configured to expand when the implant increases in temperature, such as by absorbing heat energy from the surrounding tissue or other biological material of the body after implantation in the sinus cavity. For example and without limitation, some embodiments of the sinus implant 100, 200, 300 can be configured to be implanted at a first temperature and a first size of the implant and be configured to expand to a second size of the implant when the implant is heated to a second temperature. In some embodiments, the first temperature can be room temperature, which can be 70 degrees Fahrenheit, or any temperature from 70 degrees or approximately 70 degrees Fahrenheit to 72 degrees or approximately 72 degrees Fahrenheit or any temperature from 68 degrees or approximately 68 degrees Fahrenheit to 74 degrees or approximately 74 degrees Fahrenheit. In some embodiments, the second temperature can be body temperature, which can be 98.6 degrees Fahrenheit, or any temperature from 97 degrees or approximately 97 degrees Fahrenheit to 99 degrees or approximately 99 degrees Fahrenheit.

In some embodiments, the sinus implant 100, 200, 300 can be configured to expand in volumetric size by 10% when the sinus implant is implanted in the body or otherwise heated up from the first temperature to the second temperature. Some embodiments of the sinus implant 100, 200, 300 can be made from a material that is configured to expand when the sinus implant increases in temperature, for example and without limitation, when the sinus implant is moved from a room temperature environment to a physiological temperature environment within the sinus cavity. The expansion may alternatively be triggered by contact with a fluid or other environmental change. In some embodiments, the sinus implant 100, 200, 300 can be configured to expand in volumetric size by 5%, approximately 5%, or 5% or more, or by 10%, approximately 10%, or 10% or more, or when the sinus implant is implanted in the body or otherwise heated up from a first temperature to a second temperature. In some embodiments, the sinus implant 100, 200, 300 can be configured to expand in volumetric size from 5% or approximately 5% to 20%, approximately 20%, or 20% or more, or when the sinus implant is implanted in the body or otherwise heated up from a first temperature to a second temperature.

Expansion of sinus implant 100, 200, 300 of some embodiments can aid in more completely filling in the sinus cavity and/or recesses or micro-spaces in the cavity when the sinus implant is implanted in the sinus cavity. Other benefits of such expansion may include more effectively occupying dead space and/or more effectively obliterating air in dead space. This obliteration can aid in preventing sinus related complications. For example and without limitation, complications that can be prevented include cerebrospinal fluid leak, meningitis, sinusitis, and/or mucocele development. Furthermore, some embodiments of the sinus implant 100, 200, 300 can be configured to exert outward pressure on surrounding material when a sinus implant 100 is inserted into a cavity such as a frontal sinus cavity, which can aid in buttressing.

Any embodiments of the sinus implant 100, 200, 300 can be configured to be manufactured by 3D printing. Some embodiments of the sinus implant 100, 200, 300 can be configured to be manufactured by molding or by any other suitable manufacturing method or technique.

### Additional Details

In some embodiments, the sinus implant can be implemented by exposing a frontal sinus during a craniotomy procedure. The device can be implanted in the frontal sinus and its recesses after the frontal sinus is stripped of mucosa. This is one example of a rapid method of repair and obliteration of the frontal sinus. In other methods, the implant may be deployable through an endonasal or craniofacial approach or technique, possible using endoscopy. In some embodiments, the sinus implant can be sterilized before the sinus implant is implanted in the frontal sinus. The sinus implant can be sterilized ultraviolet (UV) light, gamma radiation, ethylene oxide, electron beams, and/or an autoclave.

The main body of any implant embodiments disclosed herein, including without limitation, any embodiments of the implants 100, 200, 300 can be approximately ellipsoid shaped, spheroid shaped, oblong, ovular, and/or any other curved or elongated shape. In some embodiments, a second portion of the implant may extend from the main body so that the implant has a seamless, continuous, and/or contoured surface. The protrusions of any implant embodiments disclosed herein, including without limitation, any embodiments of the implants 100, 200, 300 can extend outward from the main body so a transition between the main body and the protrusions is a continuous and/or a smooth or seamless transition. In some embodiments, the protrusions can extend outward from the main body so the implant has seamless, continuous, and/or contoured surface (i.e., without sharp edges, irregularities, or abrupt changes in shape). In some embodiments, the surface of the implant can gradually slope from the main body to the protrusions. In some embodiments, the protrusions can be elevated from the main body. In some embodiments, the second portion and/or the protrusions may extend from the main body so the implant comprises a monolithic structure. In some embodiments, the protrusions can be bulges in the surface of the implant. In some embodiments, the protrusions can be convex portions of the surface of the implant.

While certain embodiments of the inventions have been described, these embodiments have been presented by way of example only, and are not intended to limit the scope of the disclosure. Indeed, the novel methods and systems described herein can be embodied in a variety of other forms. Furthermore, various omissions, substitutions and changes in the systems and methods described herein can be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, can be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features can be described above as acting in certain combinations, one or more features from a claimed combination can, in some cases, be excised from the combination, and the combination can be claimed as a subcombination or variation of a subcombination.

Moreover, while operations can be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations can be rearranged or reordered in other implementations. Those skilled in the art will appreciate that in some embodiments, the actual steps taken in the processes illustrated and/or disclosed may differ from those shown in the figures. Depending on the embodiment, certain of the steps described above can be removed, others can be added. Furthermore, the features and attributes of the specific embodiments disclosed above can be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages can be achieved in accordance with any particular embodiment. Thus, for example, those skilled in the art will recognize that the disclosure can be embodied or carried out in a manner that achieves one advantage or a group of advantages as taught herein without necessarily achieving other advantages as can be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. can be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, in some embodiments, the terms “approximately”, “about”, “generally,” and “substantially” may refer to an amount that is within 10% of the stated value or amount. In other embodiments,, the terms “approximately”, “about”, “generally,” and “substantially” may refer to an amount that is within 5% of, within 1% of, within 0.1% of, or within 0.01% of the stated value or amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and can be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. An implant for a sinus cavity, comprising:
a main body portion;
a second body portion coupled with the main body portion, the second body portion extending at an angle away from the main body portion;
wherein:
the implant is sized and configured for implantation in the frontal sinus;
a size and a shape of the implant is similar to a size and a shape of the frontal sinus of a patient that the implant will be implanted into; and
the implant is configured to expand in situ when the implant increases in temperature after implantation of the implant in the sinus cavity.

2. The implant of claim 1, wherein the second body portion has a tapering cross-sectional shape along a length of the second body portion such that a cross-sectional size of the second body portion is smaller at a distal end portion of the second body portion than at a proximal portion of the second body portion, wherein the proximal portion of the second body portion is coupled with the main body portion.

3. (canceled)

4. The implant of claim 1, wherein the second body portion extends away from the main body portion at an angle that is from 60 degrees to 80 degrees relative to an approximate centerline of the main body portion.

5. (canceled)

6. The implant of claim 1, wherein the structure is printed in a thermoplastic polymer material comprising one or more of polyethylene, high density polyethylene (HDPE), polymethyl methacrylate (PMMA), or polylactide (PLA) or in a thermoset crosslinked polymer comprising one or more of polyethylene glycol (PEG), polydimethylsiloxane (PDMS), or polydioxanone (PDS).

7. (canceled)

8. The implant of claim 1, wherein the implant is made from an elastic or moldable putty-like material.

9. The implant of claim 1, wherein the implant is sized and shaped to fill and obliterate the frontal sinus space, or gap between the forehead, frontal bone, sinus septum, and frontal infundibulum.

10. The implant of claim 1, wherein the shape and size of the implant is based on a collection of previously analyzed CT and MRI head scans of adult males and females.

11-24. (canceled)

25. The implant of claims claim 1, wherein the implant is prism-shaped and has one or more protrusions on each of an anterior surface and a posterior surface, wherein the protrusions are trimmable by hand to better confirm the implant to a patient's sinus cavity.

26. The implant of claim 1, wherein the implant can have a tapered adjunction to fill and/or plug the frontal infundibulum and/or the frontal recess.

27. The implant of claim 1, wherein the implant is printed in different layers to have a changing gradient of hardness from a core portion of the implant to an outer surface of the implant that decreases in hardness.

28. The implant of claim 1, wherein a core portion of the implant is made from a first material and an outer portion of the implant is made from a second material that is different than the first material.

29. (canceled)

30. (canceled)

31. The implant of claim 1, wherein a core portion of the implant is harder than an outer, malleable or moldable portion of the implant.

32. The implant of claim 1, wherein the implant is PEGylated to improve a compliance of the implant for improved fit, hydrophobicity, and drug delivery applications.

33. The implant of claim 1, wherein the implant is configured to expand in situ by 5% or approximately 5% to 20% or approximately 20% by volume after implantation in a patient's sinus cavity.

34. (canceled)

35. The implant claim 1, wherein the implant is configured to expand in situ by at least 10% by volume after implantation in a patient's sinus cavity.

36. The implant of claim 1, wherein a core portion of the implant is 3D printed from a PE, HDPE, PMMA, or PLA with an elastic modulus above 500 MPa.

37. (canceled)

38. The implant of claim 1, wherein an outer portion of the implant is 3D printed from PDMS, PEG, or PDS.

39-45. (canceled)

46. The implant of claim 1, wherein the implant is configured to elute antibiotics and/or steroids or other compounds to suppress inflammation.

47-50. (canceled)

51. A method of making a sinus implant for a frontal sinus, comprising:
reviewing imaging data of a frontal sinus region from a plurality of patients;
forming a first frontal sinus implant for a first range of patients, the first frontal sinus implant having a first size and comprising:
a main body portion; and
a second body portion extending at an angle away from the main body portion;
wherein, the first frontal sinus implant is shaped to approximately match a shape of the frontal sinus.

52-59. (canceled)

60. A method of treating a frontal sinus of a patient during a craniotomy procedure, comprising:
providing an implant that has a shape that approximates a shape of the frontal sinus of the patient;
trimming or molding the implant to be positioned within the frontal sinus of the patient; and
positioning the implant in the front sinus so a portion of the implant is positioned in a frontal recess of the front sinus to plug or obliterate, or substantially plug or obliterate, the frontal sinus.

61-68. (canceled)
